(19) United States
(12) Patent Application Publication
(10) Pub. No.: US 2022/0143158 A1
(43) Pub. Date: May 12, 2022

(54) DUODENAL ADMINISTRATION OF BOTULINUM TOXIN

(71) Applicant: Vanderbilt University, Nashville, TN (US)

(72) Inventors: Naji N. ABUMRAD, Nashville, TN (US); Sinju SUNDARESAN, Nashville, TN (US); Babak BANAN, Nashville, TN (US); Charles Robb FLYNN, Nashville, OH (US); Phillip WILLIAMS, Nashville, TN (US); Cyndya SHIBAO, Nashville, TN (US); Ornella SIMO, Nashville, TN (US)

(21) Appl. No.: 17/479,381

(22) Filed: Sep. 20, 2021

Related U.S. Application Data
(60) Provisional application No. 63/080,171, filed on Sep. 18, 2020.

(51) Int. Cl. A61K 38/48; A61K 9/00; A61P 3/10

(52) U.S. Cl. CPC A61K 38/4893; A61P 3/10; A61K 9/0019

(57) ABSTRACT

A method of treating an obese subject, or a subject having type II diabetes is described. The method includes injecting a therapeutically effective amount of botulinum toxin into the duodenum of the subject. Methods of preventing the development of type II diabetes in subjects such as prediabetic subjects by injection of botulinum toxin are also described.

### CROSS-REFERENCE TO RELATED APPLICATIONS

This application claims priority from U.S. Provisional Application Ser. No. 63/080,171, filed Sep. 18, 2020, which is incorporated herein by reference.

### BACKGROUND

A devastating consequence of the global obesity epidemic is a steep rise in type 2 diabetes (T2D). Currently, about 415 million adults (1 in 11) have diabetes worldwide. In the US alone, about 30 million people are diabetic, with T2D accounting for 90-95% of all diagnosed cases, and is the 7^{th} leading cause of death. In addition to overweight or obesity, age (>45 years), genetic predisposition, and low physical activity are known to be risk factors for the development of T2D. Lifestyle interventions including eating healthy and increasing physical activity are effective, however they have been proven to be only moderately successful due to challenges of compliance and lack of sustained benefits. Bariatric surgery, widely accepted to be the most effective treatment for sustained weight loss, is associated with remarkable improvements in T2D (75% remission at 2 years, 62% at 6 years, and 51% at 12 years). Adams et al., N Engl J Med., 377(12), 1143-1155 (2017) However, these surgical procedures involve extensive physiological manipulations of the gastrointestinal (GI) tract, are expensive, and often associate with complications including hernias, gastric ulcers, bowel obstruction, emboli, and nutrient deficiencies. Thus, it is clear that a need exists for the development of an approach that would result in effective and sustainable remission of T2D.

### SUMMARY OF THE INVENTION

Botox, the commercial product using Botulinum toxin type A (BtX-A), has been in the marketplace over 5 decades. About 55% of all Botox applications constitute cosmetic purposes, while 45% are approved for medical purposes. It has been approved by the FDA for treating spasticity or muscle stiffness in children with cerebral palsy. It is also used as adjunct therapy for gastroparesis in adolescents. While the rationale for its use as therapy in these conditions is based on its actions on neuromuscular junctions and consequent relaxation of muscle motility, possible effects on GI smooth muscle, glucose homeostasis, and amelioration of insulin resistance, has not been explored. Therefore, testing its effectiveness in interfering with intestinal motility and consequent effects on glucose absorption and insulin sensitivity would be a potentially unique and novel application of the drug.

BtX-A could provide an additional treatment option for the amelioration of obesity and T2D. In particular, obese subjects who do not quality for or cannot afford bariatric surgery could opt for this relatively minimally invasive procedure to derive comparable benefits. Repeated administration may be required, since the action of the drug is reversible. Endoscopic delivery is far less invasive, relatively inexpensive, and does not alter the GI anatomy or physiology.

The present invention overcomes the difficulties encountered by either medical (e.g., failure of dietary and medical approaches for the treatment of T2D) or the surgical approaches (e.g., cost of procedures, potential for post-operative complications; malabsorption, etc.). The inventors have developed a method of delivering BtX-A endoscopically into the gastrointestinal wall of humans with established diabetes or pre-diabetes, with or without obesity with established diagnosis of T2D or pre-diabetes.

### BRIEF DESCRIPTION OF THE FIGURES

The present invention may be more readily understood by reference to the following figures, wherein:

FIGS. 1A-1E provide multiple graphs showing the effects of injections of either Saline or BtX-A (0.1-5 U/kg body weight) in the duodenal wall of mice (n=7-8 mice) on: (FIG. A)—shows detection of a representative specific SNAP-25 protein in mice duodenum at 6 weeks post-, BtX-A injection. (FIG. B)—Weight loss in diet induced obese (DIO). (FIG. C) Body composition analyses of DIO mice injected with saline or BtX-A (3 U/kg body weight) before (Week 0) and 4 and 10 weeks after injections. (FIG. D): Food intake in DIO mice injected with saline or BtX-A (3 U/kg body weight). (FIG. E) shows the weight loss in pair-fed Saline or BtX-A injected DIO mice (n=6-8 pairs)*, p<0.05; **, p<0.01.

FIGS. 2A-2E provide multiple graphs showing fasting plasma levels of glucose (FIG. 2A) and insulin (FIG. 2B) and HOMA-IR (FIG. 2C) in DIO mice injected with BtX-A (3 U/kg) or saline tested at 1-, 4-, 7- and 10-weeks post-injection. FIG. 2D depicts plasma glucose levels following an oral glucose tolerance test (OGTT) in diet-induced obese mice injected with saline or BtX-A (3U/kg) in the duodenal wall, measured after 10 days of injection (n=14, 6, 8 for baseline, post-saline, and post-BtX, respectively). FIG. 2E depicts the area under the curve (AUC) for glucose for the oral glucose tolerance test curves shown in FIG. 2D.

FIGS. 3A-3G show fasting plasma levels of glucose (3A), insulin (3B) and HOMA-IR (3C) in pair-fed mice at 1 and 4-weeks post injection. FIGS. 3D, 3E and 3F represent the plasma glucose levels in pair-fed mice pre-saline or BtX-A injections (Week 0) and after 1-week and 4-weeks post injections in pair fed mice. FIG. 3G represents the glucose AUC for the same animals in FIGS. 3D-E.

FIGS. 4A-4E provide graphs showing that BtX-A injection (3 U/kg body weight) into the duodenal wall of DIO mice improves plasma lipids (FIGS. 4A-C) and improves lipid tolerance in DIO mice following oral gavage of lipid emulsion measured 10 days after injection (FIGS. 4D-E).

FIGS. 5A-5C provide graphs showing that the Botox mediated improvements in hyperlipidemia are not entirely dependent on reduced food intake. Fasting plasma levels of triglycerides (FIG. 5A), free fatty acids (FFA-FIG. 5B) and cholesterol (FIG. 5C) were markedly decreased following Botox injections in diet obese pair-fed mice

FIGS. 6A-6D show graphs for fecal output of lipids (A), triglycerides (B), free fatty acids (C) and cholesterol (D) in Botox- and saline-injected mice pair-fed for 4 weeks (n=6-8 pairs). These depict that the Botox-mediated improvement in hyperlipidemia is related to decreased absorption of lipids from the gastrointestinal tract, confirmed in FIG. 7.

FIGS. 7A-7H show that Botox-mediated improvements in glucose and lipid tolerance are the result of reduced intestinal glucose and lipid absorption. FIGS. 7A-B and FIGS. 7G-H depict rates of glucose absorption (using ³H-glucose tracer) and oleic acid (using ³H-oleic acid tracer) in saline and Botox-injected mice. FIGS. 7C-F depict the fecal output of fat (C), triglycerides (D), free fatty acids (E) and cholesterol (F) at various weeks after injections.

FIGS. 8A-8F show the total gastrointestinal transit time in diet-induced obese mice injected with saline or BtX-A (3U/kg body weight) in the duodenal wall, measured after 4 weeks of injection (n=4-8 mice).

FIGS. 9A-9F show the average energy expenditure (A), average respiratory exchange (B), average oxygen consumption (C), distance traveled (D), proportion of time walking (E) and all locomotor movements (F) in saline- or Botox-injected mice, measured 1-week after injections (n=6-8 pairs).

### DETAILED DESCRIPTION OF THE INVENTION

The present invention provides a method of treating a subject who is either obese, pre-diabetic or having type II diabetes that includes injecting a therapeutically effective amount of botulinum toxin into the duodenum of the subject. Methods of preventing the development of type II diabetes in subjects such as prediabetic subjects by injection of botulinum toxin are also provided.

### Definitions

The terminology as set forth herein is for description of the embodiments only and should not be construed as limiting of the invention as a whole. As used in the description of the invention and the appended claims, the singular forms “a”, “an”, and “the” are inclusive of their plural forms, unless contraindicated by the context surrounding such.

A subject, as defined herein, is an animal such as a vertebrate or invertebrate organism. In some embodiments, the subject is a single celled organism such as a yeast or bacteria. In other embodiments, the subject is a mammal such as a domesticated farm animal (e.g., cow, horse, pig) or pet (e.g., dog, cat). More preferably, the subject is a human. The subject may also be a subject at risk of developing type-II diabetes.

Treat”, “treating”, and “treatment”, etc., as used herein, refer to any action decreasing the rate of obesity of a subject or providing a benefit to a subject having an obesity-related disease, such as type 2 diabetes, cardiovascular diseases, and other obesity associated diseases including improvement in the condition through lessening or suppression of at least one symptom, delay in progression of the disease, etc.

As used herein, the term “prevention” includes either preventing or decreasing the risk of developing an obesity-related disease or disorder. This includes prophylactic treatment of those having an enhanced risk of developing type II diabetes. An elevated risk represents an above-average risk that a subject will develop an obesity-related disease or disorder, which can be determined, for example, through family history or the detection of genes causing a predisposition to developing type II diabetes.

“Pharmaceutically acceptable” as used herein means that the compound or composition is suitable for administration to a subject for the methods described herein, without unduly deleterious side effects in light of the severity of the disease and necessity of the treatment.

The terms “therapeutically effective” and “pharmacologically effective” are intended to qualify the amount of each agent which will achieve the goal of decreasing disease severity while avoiding adverse side effects such as those typically associated with alternative therapies. The therapeutically effective amount may be administered in one or more doses. An effective amount, on the other hand, is an amount sufficient to provide a significant chemical effect, such as improving glucose tolerance by a detectable amount, or decreasing the rate of decrease of glucose tolerance.

Although T2D remission after bariatric surgery could initially appear to be the result of weight loss, resolution of diabetes often occurs early in the post-operative period, before significant weight loss has occurred. The mechanisms underlying this effect remain unclear. The GI tract is abundantly innervated by vagal afferent nerve fibers, neural signals from the gut are integrated in the brainstem and transmitted to forebrain areas, including the hypothalamus that forms the basis for regulation of energy and glucose homeostasis. Entry of glucose into the intestinal lumen triggers vagal afferent signaling that synergize at the nodose ganglion and are transmitted to higher brain areas via the brainstem to elicit a response. In additional to neuronal activity in vagal afferents that project directly in to the hindbrain, there is also an intestinal release of several hormones, neuropeptides, and neurotransmitters from enteroendocrine cells that act via vagal afferents to regulate glucose homeostasis. This “gut-to-brain cross-talk” forms the basis for feeding behavior, food intake, and energy homeostasis.

The vagus nerve acts largely by releasing the neurotransmitter acetylcholine (Ach) and this step is blocked by Botulinum toxin type A (BtX-A), which selectively targets cholinergic nerve endings of presynaptic motor neurons. BtX-A exerts prolonged but reversible neurotransmission blockage. The inventors hypothesized that BtX-A targeting of gut neuromuscular junctions would be an effective adjuvant therapy for T2D. This result is achieved, in part, by altering glucose metabolism and insulin sensitivity in both obese and non-obese subjects.

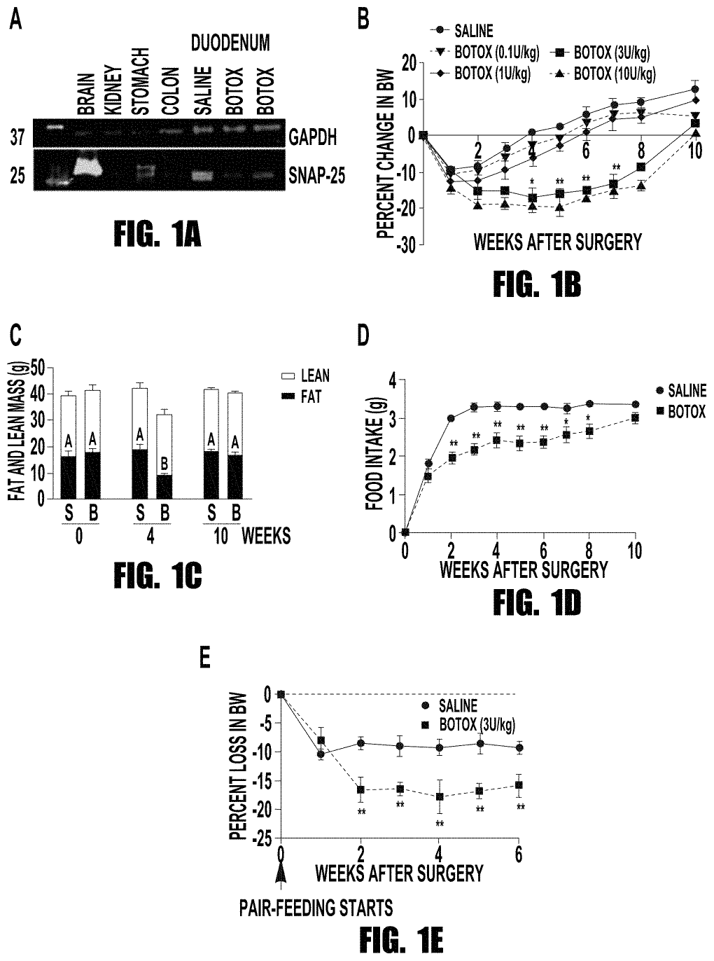

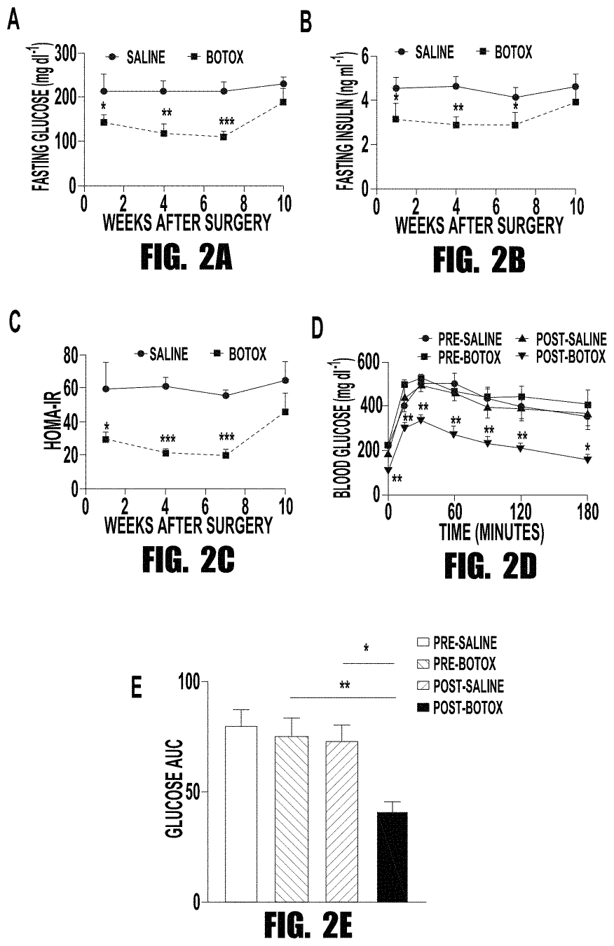

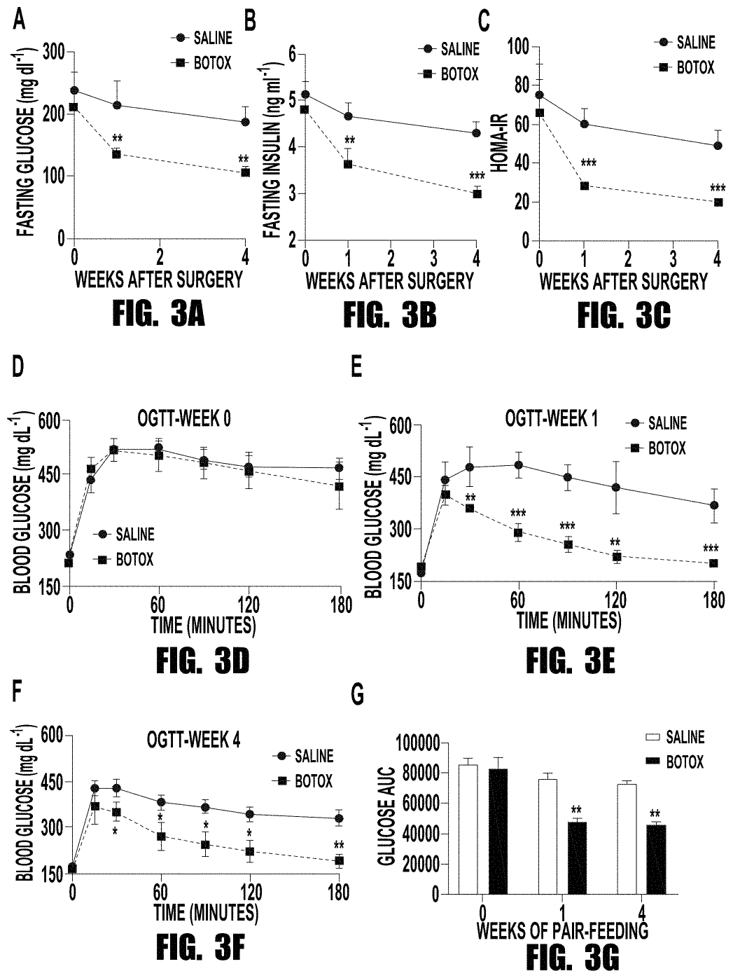

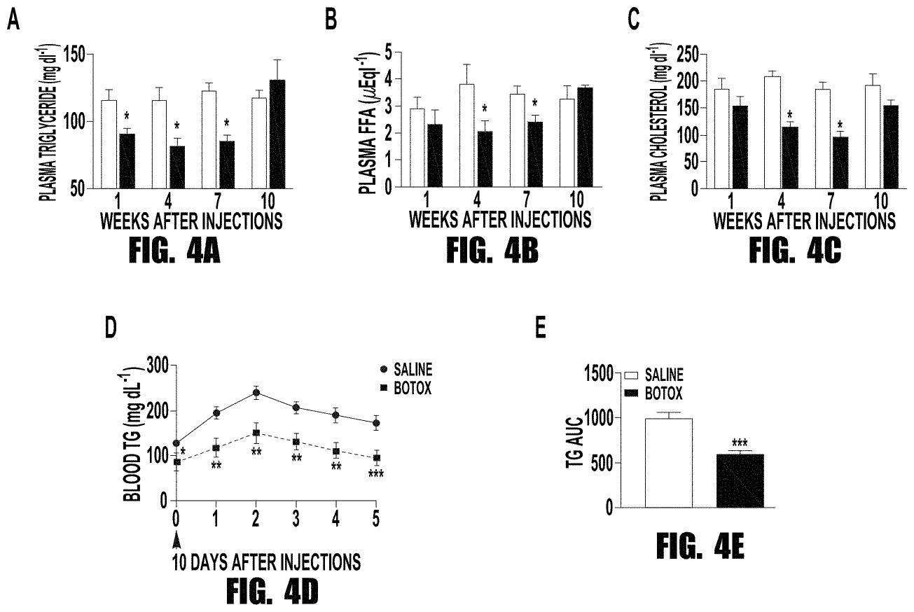

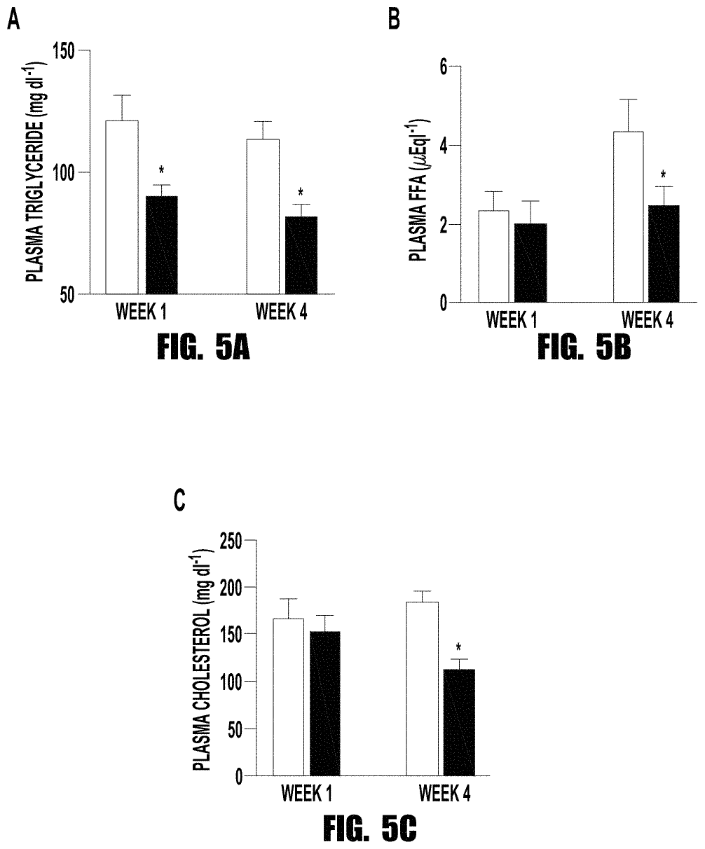

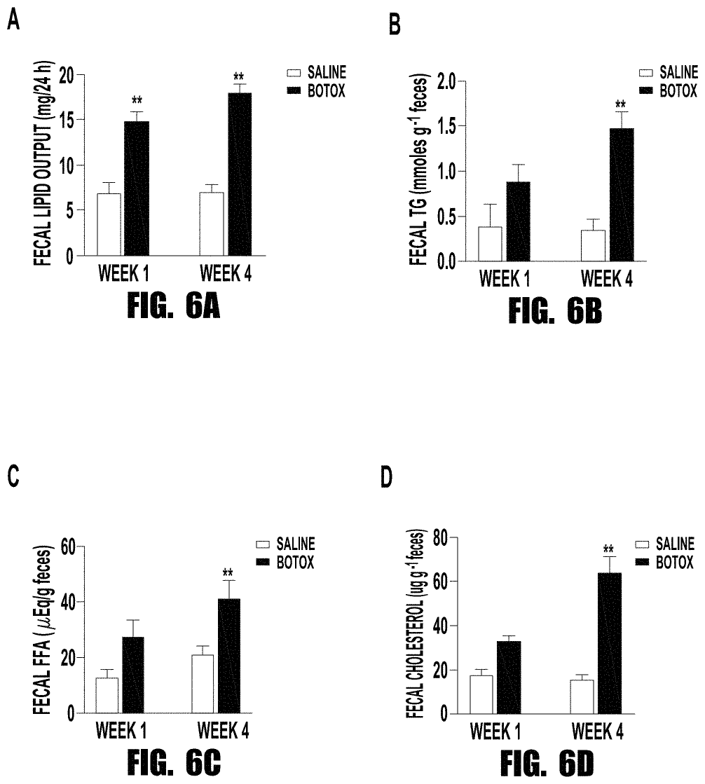

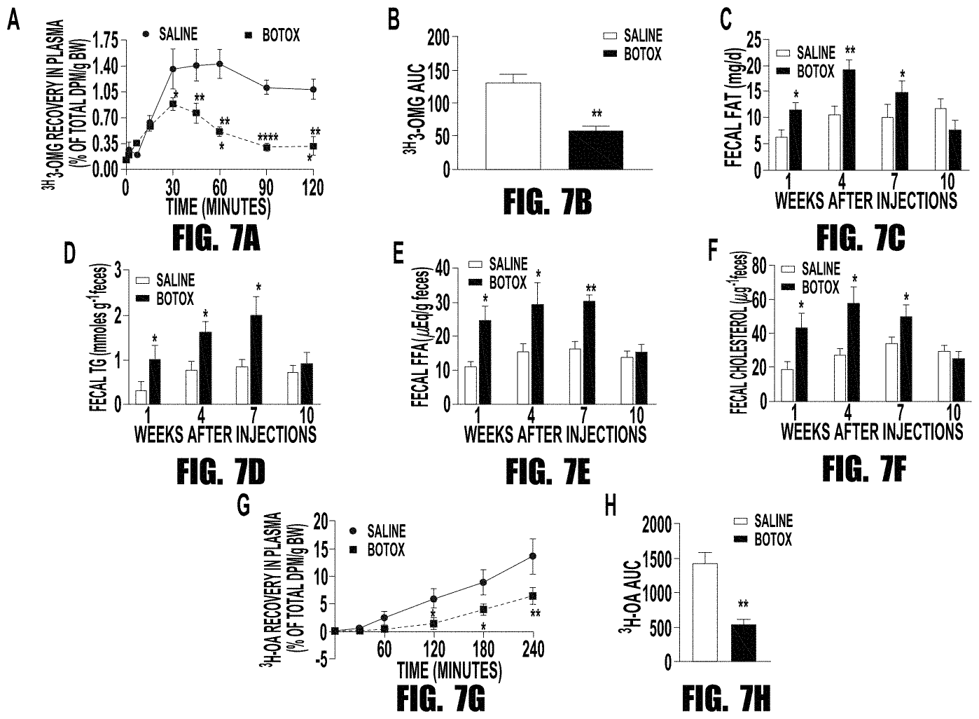

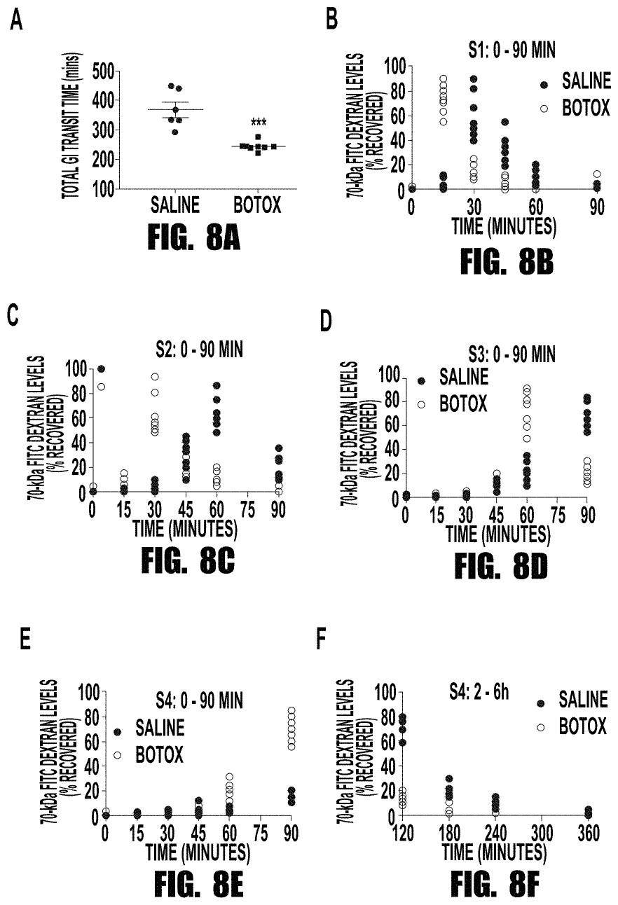

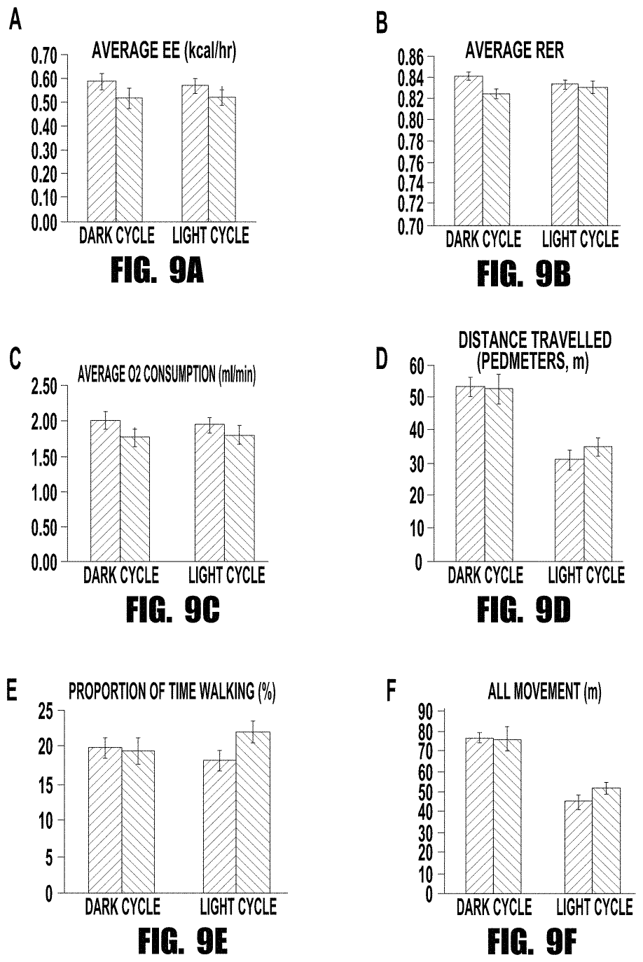

The inventors have obtained data that supports this hypothesis. This data is shown in FIGS. 1-9. These studies show that injection of BtX-A into the muscular wall of the duodenum resulted in significant improvement in glucose tolerance, indicative of improved insulin sensitivity (FIGS. 1, 2 and 3). Similarly, BtX-A injected into the muscular wall of the duodenum of mice exhibited significant reductions in plasma lipids (e.g., triglycerides, free fatty acids, and plasma cholesterol (FIGS. 4A, 4B, and 4C), and improved lipid tolerance to high fat meal (FIGS. 4D and 4E). Btx-A injected into the muscular wall of the duodenum of mice had accelerated GI transit (FIG. 8), suggesting that glucose and fat absorption via enterocytes may be reduced (FIG. 7), as crucial determinants of glucose and lipid homeostasis. FIG. 1B shows that Btx-A injection also results in a loss of body weight. The inventors have also shown that BtX-A blocks Ach release by testing the expression of SNAP-25, the upstream mediator of Ach release. BtX-A injection (3 U/kg body weight (BW)) into the duodenal wall led to a significant reduction of SNAP-25 content at this site 3 days post injection, indicating that the drug reached its target (FIG. 1A). The regulatory mechanisms coordinated GI transit are complex and involve the central nervous system (CNS), vagal afferents, enteric motor neurons, and the gastric smooth muscle cells. Normal GI rate is stimulated by contractile activity of the stomach and small intestine, which is coordinated by the CNS, the vagus nerve, and neuro-hormonal peptides. The inventors believe that BtX-A mediated alteration of GI smooth muscle motility also impedes glucose and lipid absorption, and consequently regulates post-prandial glucose and lipid (triglycerides, free fatty acids, and cholesterol) levels.

In one aspect, the present invention provides a method of treating a subject having type II diabetes. The method includes injecting a therapeutically effective amount of botulinum toxin into the duodenum of the subject.

The duodenum is the first section of the small intestine in most higher vertebrates, including mammals, reptiles, and birds. The duodenum is largely responsible for the breakdown of food in the small intestine, using enzymes. The duodenum precedes the jejunum and ileum and is the shortest part of the small intestine. In humans, the duodenum is a hollow jointed tube about 25-38 cm (10-15 inches) long connecting the stomach to the jejunum. It begins with the duodenal bulb and ends at the suspensory muscle of duodenum. The duodenum includes a smooth muscle layer, also referred to as the muscularis mucosa, as well as an epithelial layer. In some embodiments, the botulinum toxin is injected into the smooth muscle layer of the duodenum.

### Botulinum Toxin

Botulinum Toxin is a neurotoxic protein produced by Gram-positive, anaerobic bacterium Clostridium botulinum. There are seven different serotypes of botulinum toxins designated by the letters A, B, Cl, D, E, F, and G. Each serotype has more subtypes (e.g., the subtype A contains four distinct subtypes). All serotypes have similar chemical structures and are neurotoxins, with the exception of the C2 subtype. Goonetilleke A., Harris J. B, J. Neurol. Neurosurg. Psychiatry, 75:35-39 (2004). Botulinum toxin acts in the neuromuscular junction (endplate) blocking the release and the effects of acetylcholine, an acetic acid ester and choline, responsible for neurotransmission in the central nervous system (CNS) and peripheral nervous system (PNS). Cherington M., Semin. Neurol., 24:155-163 (2004).

Botulinum toxin is a zinc-dependent endoprotease that acts on vulnerable cells to cleave polypeptides that are essential for exocytosis. The general structure presents two chains of a total weight of approximately 150 kDa. The two chains are a heavy chain (100 kDa) and a light chain (50 kDa) joined by a disulfide bridge. The heavy chain is in turn constituted from the N-terminal domain and the C-terminal domain. Each botulinum toxin is initially synthesized as a double polypeptide, but the biological activity requires a post-translational proteolysis that cleaves the polypeptide in two separate portions. Rossetto et al., Nat. Rev. Microbiol., 12:535-549 (2014). In some embodiments, the botulinum toxin injected into the subject is botulinum toxin A. Commercial forms of BtX-A are marketed under the brand names Botox (onabotulinumtoxinA), Dysport/Azzalure (abobotulinumtoxinA), Xeomin/Bocouture (incobotulinumtoxinA), and Jeuveau (prabotulinumtoxinA).

### Type II Diabetes

Diabetes mellitus is a serious metabolic disease that is defined by the presence of chronically elevated levels of blood glucose (hyperglycemia). This state of hyperglycemia is the result of a relative or absolute lack of activity of insulin, which is produced and secreted by the β-cells of the pancreas. Insulin promotes glucose utilization, protein synthesis, and the formation and storage of carbohydrate energy as glycogen. Glucose is stored in the body as glycogen, a form of polymerized glucose, which may be converted back into glucose to meet metabolism requirements. Under normal conditions, insulin is secreted at both a basal rate and at enhanced rates following glucose stimulation, all to maintain metabolic homeostasis by the conversion of glucose into glycogen.

The term diabetes mellitus encompasses several different hyperglycemic states. These states include Type I (insulin-dependent diabetes mellitus or IDDM) and Type II (non-insulin dependent diabetes mellitus or NIDDM) diabetes. The hyperglycemia present in individuals with Type I diabetes is associated with deficient, reduced, or nonexistent levels of insulin which are insufficient to maintain blood glucose levels within the physiological range. Traditional treatment of Type I diabetes involves administration of replacement doses of insulin, generally by a parenteral route. The hyperglycemia present in individuals with Type II diabetes is initially associated with normal or elevated levels of insulin; however, these individuals are unable to maintain metabolic homeostasis due to a state of insulin resistance in peripheral tissues and liver and, as the disease advances, due to a progressive deterioration of the pancreatic β-cells which are responsible for the secretion of insulin. Thus, initial therapy of Type II diabetes may be based on diet and lifestyle changes augmented by therapy with oral hypoglycemic agents such as sulfonylureas. Insulin therapy is often required, however, especially in the latter states of the disease, in order to produce some control of hyperglycemia and minimize complications of the disease.

Diabetes mellitus is characterized by recurrent or persistent high blood sugar. The presence of diabetes in a subject can therefore be identified by a random venous plasma glucose concentration of ≥11.1 mmol/l; a fasting plasma glucose concentration ≥7.0 mmol/l (whole blood ≥6.1 mmol/1) and/or a two-hour plasma glucose concentration >11.1 mmol/l two hours after 75 g anhydrous glucose in an oral glucose tolerance test (OGTT). Glycated hemoglobin levels ≥48 mmol/mol, or ≥6.5 DCCT% (Diabetes Control and Complications Trial) can also be used to diagnose diabetes mellitus.

Insulin resistance is a pathological condition in which cells fail to respond normally to insulin. Cells senses insulin through insulin receptors, with the signal propagating through the PI3K/AKT signaling pathway. When the body produces insulin under conditions of insulin resistance, the cells are resistant to the insulin and are unable to use it as effectively, leading to high blood sugar. Insulin resistance can be measured using the oral glucose tolerance test, or using the “hyperinsulinemic euglycemic clamp,” which measures the amount of glucose necessary to compensate for an increased insulin level without causing hypoglycemia. Insulin resistance plays a key role in the development of type II diabetes mellitus.

The method of treating type II diabetes in a subject using Botulin toxin administration can also include administering insulin or an additional antidiabetic agent to the subject. Insulin is a peptide hormone produced by beta cells of the pancreatic islets. The structure and amino acid sequence of insulin is known to those skilled in the art. Insulin, as used herein, includes insulin, effective insulin fragments, insulin analogs such as Humalog, and insulin produced by various species. For example, bovine insulin differs from human insulin by only 3 amino acids, while porcine insulin differs from human insulin by only a single amino acid, and both can be used therapeutically in place of human insulin. The insulin can be obtained from an animal source, or can be produced recombinantly.

A variety of agents suitable for anti-diabetic therapy are known. Examples of types of non-insulin drugs for treating diabetes include sensitizers, secretagogues, α-glucosidase inhibitors, and glycosurics. Examples of specific non-insulin drugs for treating diabetes include biguanides, metformin, phenformin, buformin, thiazolidinediones (e.g., rosiglitazone, pioglitazone, and troglitazone), sulfonylureas (e.g., tolbutamide, acetohexamide, tolazamide, chlorpropamide, glipizide, glibenclamide, glimepiride, gliclazide, glyclopyramide, and gliquidone), repaglinide, nateglinide, miglitol, acarbose, voglibose, exenatide, liraglutide, taspoglutide, lixisenatide, vildagliptin, sitagliptin, saxagliptin, linagliptin, alogliptin, septagliptin, teneligliptin, and gliflozin.

Another aspect of the invention provides a method of preventing the development of type II diabetes in a subject having an increased risk of developing type II diabetes (e.g., a pre-diabetic subject), comprising injecting an effective amount of botulinum toxin into the duodenum of the subject. The method of prevention includes all of the features of the method of treating diabetes described herein, except that the method is used to decrease the likelihood that type II diabetes will develop, rather than treating type II diabetes that already exists. For example, in some embodiments the botulinum toxin is botulinum toxin A.

Methods of preventing diabetes are more frequently used for subjects who have an increased risk of developing diabetes. For example, it may be used in subjects who are pre-diabetic. Pre-diabetes is diagnosed when a person has either Impaired Fasting Glucose, or Impaired Glucose Tolerance. The two main tests used in diagnosing pre-diabetes are the morning fasting glucose test and the two-hour oral glucose tolerance test. A person with prediabetes has a fasting blood glucose level between 100 and 125 mg/dl.

### Obesity Treatment

Another aspect of the invention provides a method of treating an obese subject. The method of treating an obese subject includes the step of injecting a therapeutically effective amount of botulinum toxin into the duodenum of the subject. The method can include administering various types of botulinum toxin to a subject using any of the methods and formulations described herein. Treating obesity decreases the body weight of the subject being treated.

Obesity is a medical condition in which excess body fat has accumulated to the extent that it may have a negative effect on health. Obesity is generally defined as occurring when a subject's body weight is at least 20% greater than its optimal body weight. Obesity treatment is primarily provided for human subjects, but various animals, and in particular pets such as cats and dogs, can also be obese. A human subject can be considered as obese if the body mass index is at least 25 kg/m². In the present invention, an obese human subject may have a body mass index of at least 25 kg/m², at least 26 kg/m², at least 27 kg/m², at least 28 kg/m², at least 29 kg/m², at least 30 kg/m² or at least 31 kg/m². In some embodiments, a subject having a BMI ranging from about 25 kg/m² to about 31 kg/m² is considered to be obese. Treatment of obesity results in a decrease in the body weight of the subject, preferably to an extend that the subject is no longer obese.

### Formulation and Administration

The present invention provides a method for treating obesity or type II diabetes in a subj ect by injecting botulinum toxin in a pharmaceutical composition. Examples of pharmaceutical compositions include those that can be injected, such as intravenous, intramuscular, subcutaneous, or intraperitoneal administration, and generally involves providing the botulinum toxin formulated together with a pharmaceutically acceptable carrier.

For intravenous, intramuscular, subcutaneous, or intraperitoneal administration, the compound may be combined with a sterile aqueous solution which is preferably isotonic with the blood of the recipient. Such formulations may be prepared by dissolving solid active ingredient in water containing physiologically compatible substances such as sodium chloride, glycine, and the like, and having a buffered pH compatible with physiological conditions to produce an aqueous solution, and rendering said solution sterile. Injection is typically made to a specific region or portion of the duodenum.

Formulations suitable for parenteral administration can comprise a sterile aqueous preparation of the active compound which is preferably made isotonic. Preparations for injections may also be formulated by suspending or emulsifying the compounds in non-aqueous solvent, such as vegetable oil, synthetic aliphatic acid glycerides, esters of higher aliphatic acids or propylene glycol.

The dosage form and amount can be readily established by reference to known treatment or prophylactic regiments. The amount of therapeutically active compound that is administered and the dosage regimen for treating a disease condition with the compounds and/or compositions of this invention depends on a variety of factors, including the age, weight, sex, and medical condition of the subject, the severity of the disease, the route and frequency of administration, and the particular compound employed, the location of the unwanted proliferating cells, as well as the pharmacokinetic properties of the individual treated, and thus may vary widely. The dosage will generally be lower if the compounds are administered locally rather than systemically, and for prevention rather than for treatment. Such treatments may be administered as often as necessary and for the period of time judged necessary by the treating physician. In some embodiments, a plurality of injections are provided to the subject at 3-to-6-month intervals. One of skill in the art will appreciate that the dosage regime or therapeutically effective amount of the inhibitor to be administrated may need to be optimized for each individual. In some embodiments, 1-5 U/kg per body weight, 1-10 U/kg per body weight, 5-10 U/kg per body weight, or 5-20 U/kg per body weight of botulinum toxin are injected into the subject.

In some embodiments, the botulinum toxin is injected endoscopically. Endoscopy is a diagnostic and medical procedure which allows to examine the interior of a hollow organ or cavity of the body by means of an instrument called endoscope, without employing invasive surgery. In particular, endoscopic procedures are widely applied in the gastrointestinal tract, both for diagnostic purposes and for small interventions. Endoscopy is carried out using a device called an endoscope that includes an illuminated usually optic fiber flexible or rigid tubular instrument for visualizing the interior of a hollow organ or part and includes one or more working channels to enable passage of instruments, such as an endoscopic injection needle.

An Example has been included to more clearly describe a particular embodiment of the invention and its associated cost and operational advantages. However, there are a wide variety of other embodiments within the scope of the present invention, which should not be limited to the particular examples provided herein.

### EXAMPLE

### Example 1

### Investigating Effects of BOTOX on Weight Loss and Glucose Tolerance in Obese, Type 2 Diabetic Subjects

Obese subjects (body mass index≥30 kg/m²) with prediabetes or type 2 diabetes will be recruited by solicitation flyers, advertisements, and mass emails. Subjects who do not qualify for bariatric surgery, or subjects who are awaiting insurance approval for a bariatric procedure at the Center of Surgical Weight Loss at Vanderbilt University Medical Center will also be contacted via emails and telephone calls. Subjects will be directed to contact the researcher (Drs. Abumrad, Shibao, Flynn and Sundaresan) directly for more information. The recruiter will review inclusion and exclusion criteria and explain the study procedures, duration of the study and potential risks. If the subject indicates interest, screening will be performed to determine eligibility.

Subjects will undergo an initial screen by the study coordinator or research staff to assess eligibility and ability to comply with the study requirements. Prior to each study visit, subjects on oral anti-diabetic medications will be asked to discontinue these medications 4 days prior to their study visit. Diabetic subjects will be instructed to monitor their pre-prandial blood glucose during this time and to contact the study physician if their blood glucose levels are greater than 250 mg/dL for two consecutive readings. The study physician may instruct the subject to initiate short-term insulin therapy or resume their oral anti-diabetic medications; in either case the subject will be excluded from the study. Subjects will be instructed to maintain their usual diet and physical activity levels for 1 week prior to each study visit and to arrive fasted for every visit (only water after dinner).

### Study Procedures

The subjects will be recruited for 5 study visits. After informed, written consent is obtained, subjects will be admitted to the Clinical Research Center.

### Study Visit 1:

Day 1: Subjects will arrive after an overnight fast. They will undergo standard physical examination; anthropometric measurements (height, weight, waist, and hip circumference) perform body composition assessment via dual-energy x-ray absorptiometry imaging will be recorded. Baseline glucose tolerance will be assessed by an oral glucose tolerance test. Subjects will be handed a visual analog scale questionnaire designed to capture their perceived hunger and satiety sensations, food preferences, cravings, and feeding behavior prior to intervention. The questions will be explained by the researcher and subjects will be instructed to bring in the completed questionnaire on their second visit. Subjects will be provided snacks and asked to return at 7 pm for the nutrient sensing test to be performed the following day. The subject will be fed a standardized meal and restricted to water after 8:00 pm.

Day 2: Blood will be drawn for determination of fasting plasma insulin, and gut hormones including ghrelin, Gastric inhibitory peptide, glucagon like peptide-1, pancreatic polypeptide, and Peptide YY. Subject will then consume a standardized 250 kcal liquid mixed meal containing 40 g carbohydrates, 6 g fat, and 9 g protein within 10 minutes. Blood will be drawn at 15, 30, 60, and 120 minutes after consumption following which subject will be discharged.

Study Visit 2 (within 2 Weeks After the First Visit):

Subjects will undergo esophagogastroduodenoscopy procedure for the delivery of BOTOX to the duodenal wall, to be performed by Dr. Patrick Yachimski at the Vanderbilt Gastrointestinal Endoscopy Suite. The investigators have been exempted from Investigational New Drug (IND) regulation for the proposed testing by the FDA.

Subjects will be monitored for at least two hours and discharged with instructions for follow-up and contact information of the physicians' team (Drs. Abumrad, Yachimski).

Study Visits 3-5 (1, 3, and 6 months after endoscopy): On every study visit, body weight, body composition, food intake, and feeding behavior will be recorded and post-absorptive glucose tolerance will be performed. Nutrient sensing test will be repeated at visit 3.

Oral Glucose Tolerance Test (Study visits 1 and 3-5): Day 0: Subjects will be fasting overnight (and restricted to water only after 8 pm). Day 1: Blood will be drawn for determining fasting blood glucose levels. At 8:00 am subjects will drink a solution of 75 grams dextrose in 300 ml of water in 10 minutes. Blood will be drawn at 15, 30-, 45-, 60-, and 120-min post ingestion. The subject will be fed a standardized snack and discharged around noon.

Endoscopic delivery of BOTOX (Study Visit 2): Subjects will arrive fasted at the GI Suite in the main hospital at Vanderbilt University for the procedure. Under the supervision of an anesthesiologist, subjects will be given combination of intravenous medications, so they fall asleep. Dr. Yachimski will then pass the endoscope, a long, flexible tube with light, video camera and channel for small instruments including syringes through the esophagus and stomach, pylorus into the junction of the 1st and 2nd parts of the duodenum. Botox (100 units dissolved in 200 μL of sterile, preservative-free 0.9% Sodium Chloride) will be injected along the medial (mesenteric) border, into the duodenal muscle wall.

Post endoscopy Care: Following completion of endoscopy procedure and emergence from anesthesia, subjects will be monitored in the dedicated post-anesthesia care unit prior to discharge. Subjects will be provided written instructions regarding potential signs and symptoms of adverse events, including fever, pain, bleeding, and muscle weakness. They will be provided the physician call number for any questions or issues that may arise.

Nutrient sensing test (study visit 1 and 3): Day 1: After oral glucose tolerance test subjects return at 7:00 pm. They will be fed a standardized meal and fasted overnight (restricted to water after 8:00 pm). Day 2: At 8:00 am, blood will be drawn for determination of fasting plasma insulin and gut hormones stated above. Subject will then consume a standardized 250 kcal liquid mixed meal containing 40 g carbohydrates, 6 g fat, and 9 g protein to be administered over a 10-min period. Blood will be drawn 15, 30, 60, and 120 minutes later. At the end of the study, subject will be provided a standardized meal/snack and discharged.

This study began in April 2021, and is expected to be complete by the end of October 2022. See ClinicalTrials.gov Identifier NCH03991299.

The complete disclosure of all patents, patent applications, and publications, and electronically available materials cited herein are incorporated by reference. The foregoing detailed description and examples have been given for clarity of understanding only. No unnecessary limitations are to be understood therefrom. In particular, while various theories are presented describing possible mechanisms through with the compounds are effective, the compounds are effective regardless of the particular mechanism employed and the inventors are therefore not bound by theories described herein. The invention is not limited to the exact details shown and described, for variations obvious to one skilled in the art will be included within the invention defined by the claims.

What is claimed is:

1. A method of treating a subject having type II diabetes comprising injecting a therapeutically effective amount of botulinum toxin into the duodenum of the subject.

2. The method of claim 1, wherein the botulinum toxin is botulinum toxin A.

3. The method of claim 1, wherein the botulinum toxin is injected into the smooth muscle layer of the duodenum.

4. The method of claim 1, wherein the botulinum toxin is injected endoscopically.

5. The method of claim 1, wherein the subject is obese.

6. The method of claim 1, wherein a plurality of injections are provided to the subject at 3- to-6-month intervals.

7. The method of claim 1, further comprising administering an antidiabetic agent to the subject.

8. The method of claim 1, wherein 1-10 U/kg of botulinum toxin are injected into the subject.

9. A method of preventing the development of type II diabetes in a pre-diabetic subject, comprising injecting an effective amount of botulinum toxin into the duodenum of the subject.

10. The method of claim 9, wherein the botulinum toxin is botulinum toxin A.

11. The method of claim 9, wherein the botulinum toxin is injected into the smooth muscle layer of the duodenum.

12. The method of claim 9, wherein the botulinum toxin is injected endoscopically.

13. The method of claim 9, wherein the subject is obese.

14. The method of claim 9, wherein a plurality of injections are provided to the subject at 3-to-6-month intervals.

15. The method of claim 9, wherein 1-10 U/kg of botulinum toxin are injected into the subject.

16. A method of treating an obese subject comprising injecting a therapeutically effective amount of botulinum toxin into the duodenum of the subject.

17. The method of claim 16, wherein the botulinum toxin is botulinum toxin A.

18. The method of claim 16, wherein the botulinum toxin is injected into the smooth muscle layer of the duodenum.

19. The method of claim 16, wherein the botulinum toxin is injected endoscopically.

20. The method of claim 16, wherein a plurality of injections are provided to the subject at 3- to-6-month intervals.

21. The method of claim 16, wherein 1-10 U/kg of botulinum toxin are injected into the subject.
